(19) United States
(12) Patent Application Publication
(10) Pub. No.: US 2005/0100615 A1
(43) Pub. Date: May 12, 2005

(54) Process for controlling water and electrolyte balance and acid-base equilibrium in human body

(75) Inventor: Hidehiro Yamazaki, Shizuoka (JP)

Correspondence Address: BURNS DOANE SWECKER & MATHIS L L P, POST OFFICE BOX 1404, ALEXANDRIA, VA, 22313-1404, US

(73) Assignee: SHIMIZU PHARMACEUTICAL CO., LTD.

(21) Appl. No.: 10/665,373

(22) Filed: Sep. 22, 2003

(30) Foreign Application Priority Data
Nov. 1, 2002 (JP) 2002-319380

(51) Int. Cl. A61K 31/70; A61K 31/19; A61K 33/14

(52) U.S. Cl. 424/677; 424/439; 424/717; 514/23; 514/574

(57) ABSTRACT

The present invention relates to process for controlling water and electrolyte balance and acid-base equilibrium, and particularly to process for controlling water and electrolyte balance and acid-base equilibrium supervening metabolic acidosis due to burn injury, hemorrhagic shock, multiple organ failure, systemic inflammatory response syndrome (SIRS), and so on. By administering the preparation containing sodium bicarbonate as an alkalizing agent of the present invention, the acidosis correction effect is exhibited immediately after the start of the infusion and disappeared quickly by demedication. And therefore, the preparation of the present invention can be administered safely without inducing metabolic alkalosis during infusion and alkalosis after the infusion. The preparation of the present invention also has no problem with hypernatremia. The controlling water and electrolyte balance and acid-base equilibrium, and particularly to process for controlling water and electrolyte balance can be done by administering the preparation containing bicarbonate at a rate of 2 to 60 mL/kg/hour.

### TECHNICAL FIELD

The present invention relates to a process for controlling water and electrolyte balance and acid-base equilibrium.

### BACKGROUND ART

It is ideal method to adjust a pH of an infusion for human body by bicarbonate (hereinafter referred to as “HCO₃⁻”, if necessary) because pH of extracellular fluid is adjusted by bicarbonate in the body. However, since bicarbonate produces insoluble salts in combination with calcium ion and/or magnesium ion in water, the combination of these components is contraindicated, and it has been difficult to prepare the stable pharmaceutical preparation solution containing these components all together (Mitsuro Nakano & Humio Yamashita, “Complex Electrolytes Solution In the View of Pediatrics”, Clinical Water Electrolytes, Vol.3, NO.3, pp. 217-220 (March 1985,); Shouzou Koshikawa, “Bicarbonate and Phosphate”, Saishin-Igaku, Vol.26 NO.2, pp. 274-280 (February 1971,); Shouzou Koshikawa & Kimihiro Takayama, “Sodium bicarbonate Used in Fluid Therapy”, Clinical Water Electrolytes, Vol.5 NO.3, pp. 239-245 (March 1986,).

To improve the matter, it had found that sodium lactate was metabolized in the body to produce equimolar bicarbonate, and thus the preparation containing sodium lactate instead of sodium bicarbonate has been proposed. As a result, a pharmaceutically stable lactated Ringer's solution containing sodium lactate has been provided, and widely used as a first choice for the fluid replacement of the extracellular fluid in surgical operation (For example: Mitsuro Nakano & Humio Yamashita, “Complex Electrolytes Solution In the View of Pediatrics”, Clinical Water Electrolytes, Vol.3, NO.3, pp. 217-220 (March 1985,)).

However, the capability of lactic metabolism is insufficient when decrease of hepatic blood flow is observed in hepatic disease or shock, and reduction of alkalization effect or accumulation of lactic acid has been presented as problems.

To solve these problems, acetated Ringer's solution containing sodium acetate was developed. Sodium acetate is metabolized in the whole body including skeletal muscles to produce equimolar bicarbonate. The lactated Ringer's solution and the acetated Ringer's solution are presently used in anesthesiology, surgery, medical emergency center, and so on (For example: Mitsuro Nakano & Humio Yamashita, “Complex Electrolytes Solution In the View of Pediatrics”, Clinical Water Electrolytes, Vol.3, NO.3, pp. 217-220 (March 1985,); Shouzou Koshikawa, “Bicarbonate and Phosphate”, Saishin-Igaku, Vol.26 NO.2, pp. 274-280 (February 1971,).

Further, a commercially available alkalizing agent such as high concentration sodium bicarbonate or sodium lactate solution is commonly injected intravenously in the case of metabolic acidosis such as burn injury, hemorrhagic shock, multiple organ failure, systemic inflammatory response syndrome (SIRS), and so on. However, because the commercially available high concentration sodium bicarbonate solution is consisting by 7% and 8.4% of sodium bicarbonate solution, intercellular acidosis may happen in case of rapid injection of large amount of them due to the abrupt increase of osmotic pressure of extracellular fluid, leading to increase in extracellular fluid, and resulting in circulation of extracellular fluid. Under insufficient of the respiratory management, generation of large amount of carbon dioxide in blood may cause intracellular acidosis. Further, it involves the risk of hypernatremia with simultaneous administration of sodium ion (For example: Shouzou Koshikawa & Kimihiro Takayama, “Sodium bicarbonate Used in Fluid Therapy”, Clinical Water Electrolytes, Vol.5 NO.3, pp. 239-245 (March 1986, ).

### DISCLOSURE OF INVENTION

The present invention relates to process for controlling the water and electrolyte balance and acid-base equilibrium, and particularly to process for controlling water electrolyte balance and acid-base equilibrium supervening metabolic acidosis due to burn injury, hemorrhagic shock, multiple organ failure, systemic inflammatory response syndrome (SIRS), and so on.

As a result of diligent research by the present inventors, it is newly discovered that the process for controlling the water and electrolyte balance and acid-base equilibrium, by administering the preparation stably containing sodium bicarbonate without observation of any precipitation for long period of time.

Namely, the preparation containing sodium bicarbonate as alkalizing agent can provide bicarbonate ion directly unlike the sodium lactate or the sodium acetate which is metabolized to produce bicarbonate ion, and therefore this preparation is able to control the acidosis correction effect quickly.

It is well known that the administration of organic acid salt, such as sodium lactate or sodium acetate, may induce metabolic alkalosis because of bicarbonate production by metabolization after the completion of the administration thereof. Contrary, there is no anxiety about metabolic alkalosis by administration of the preparation containing sodium bicarbonate, because the acidosis correction effect disappears quickly. Further, the preparation of the present invention containing bicarbonate, containing electrolytes in a balanced manner, does not induce hypernatremia.

### ACCORDINGLY THE PRESENT INVENTION PROVIDES

(1) a method for controlling water and electrolyte balance and acid-base equilibrium, comprising administering continuously a preparation containing 130 to 145 mEq/L of sodium ion, 2 to 5 mEq/L of potassium ion, 20 to 35 mEq/L of bicarbonate ion, 90 to 130 mEq/L of chloride ion,2 to 5 mEq/L of calcium ion, 0.5 to 2.5 mEq/L of magnesium ion, 1 to 7 mEq/L of citrate ion and 0 to 5 g/L of glucose, at a rate of 2 to 60 mL/kg/hour;

(2) a method for controlling water and electrolyte balance and acid-base equilibrium, comprising adjusting the infusion speed or demedication of the preparation according to (1), by observing a data f blood gas analysis as index parameter;

(3) a method according to (2), wherein the infusion speed is adjusted n order to maintain a plasma bicarbonate concentration to be in a range f 22 to 26 mEq/L;

(4) a method according to (1) to (3) for controlling water and electrolyte balance and acid-base equilibrium of a patient with metabolic acidosis;

(5) a method according to (1) to (3) for controlling water and electrolyte balance and acid-base equilibrium of a patient with burn injury;

(6) a method according to (1) to (3) for controlling water and electrolyte balance and acid-base equilibrium of a patient with hemorrhagic shock;

(7) a method according to (1) to (3) for controlling water and electrolyte balance and acid-base equilibrium in of a patient with multiple organ failure;

(8) a method according to (1) to (3) for controlling water and electrolyte balance and acid-base equilibrium of a patient with systemic inflammatory reaction syndrome;

(9) a method according to (1) to (3) for controlling water and electrolyte balance and acid-base equilibrium of a patient under the operation and post operative patient;

(10) a method as according to (1) to (3) for controlling water and electrolyte balance and acid-base equilibrium of a patient with dehydration;

(11) a controlling agent of water and electrolyte balance and acid-base equilibrium, comprises containing 130 to 145 mEq/L of sodium ion, 2 to 5 mEq/L of potassium ion, 20 to 35 mEq/L of bicarbonate ion, 90 to 130 mEq/L of chloride ion, 2 to 5 mEq/L of calcium ion, 0.5 to 2.5 mEq/L of magnesium ion, 1 to 7 mEq/L of citrate ion and 0 to 5 g/L of glucose;

(12) a controlling agent according to (11), wherein said agent is administered at a rate of 2 to 60 mL/kg/hour to maintain a plasma bicarbonate concentration to be in a range of 22 to 26 mEq/L. (13) a controlling agent according to (11) or (12), wherein a source of citrate ion is sodium citrate and pH of the agent is adjusted to 6.5 to 7.4 by carbon dioxide gas; and

(14) a controlling agent according to (11) to (13), wherein said agent is filled in the carbon dioxide gas permeable plastic container sealed with gas un-permeable film, or in gas un-permeable container.

### BRIEF DESCRIPTION OF DRAWINGS

FIG. 1 shows the changes of blood pH in rabbit hemorrhagic shock model of Example 1.

FIG. 2 shows the changes of plasma bicarbonate concentration in rabbit hemorrhagic shock model of Example 1.

FIG. 3 shows the changes of blood base excess (hereinafter, referred to as “BE”) in rabbit hemorrhagic shock model of Example 1.

FIG. 4 shows the changes of blood pH in rabbit partial hepatectomy model of Example 1.

FIG. 5 shows the changes of plasma bicarbonate concentration in rabbit partial hepatectomy model of Example 1.

FIG. 6 shows the changes of blood BE in rabbit partial hepatectomy model of Example 1.

FIG. 7 shows the changes of plasma bicarbonate concentration in dog hemorrhagic shock model of Example 2.

FIG. 8 shows the changes of blood BE in dog hemorrhagic shock model of Example 2.

FIG. 9 shows the changes of plasma bicarbonate concentration in rabbit partial hepatectomy model of Example 2.

FIG. 10 shows the changes of blood BE in rabbit partial hepatectomy model of Example 2.

### BEST MODE FOR CARRYING OUT THE INVENTION

The present invention provides the process for controlling water and electrolyte balance and acid-base equilibrium by means of continuous infusion of the preparation containing bicarbonate and other electrolytes in a balanced manner, at a rate of 2 to 60 mL/kg/hour, preferably 5 to 20 mL/kg/hour, and more preferably 5 to 15 mL/kg/hour. The preparation of the present invention supplies bicarbonates per se to the human body, and therefore, the prompt exhibition of the acidosis correction effect can be obtained. In addition, this effect disappears quickly.

The water and electrolyte balance and acid-base equilibrium can be controlled by changing the infusion speed or demedication of the preparation, by observing a data of blood gas analysis as an index parameter.

That is, the rate of the infusion speed of the preparation is adjusted or the infusion is terminated in order to maintain the blood pH being in a range of 7.3 to 7.5 and the plasma bicarbonate concentration being in a range of 22 to 26 mEq/L.

By administering the preparation containing bicarbonate of the present invention, the acidosis correction effect is exhibited immediately after the start of infusion and disappeared quickly by demedication. Thus, the preparation of the present invention can be administered safely without inducing metabolic alkalosis during infusion and alkalosis after the demedication. The preparation of the present invention also has no problem of hypernatremia.

Further, the preparation of the present invention possesses the maintenance and the correction effect of magnesium concentration in extracellular fluid. During the surgical operation, blood is diluted by infusion, and then plasma magnesium concentration is decreased. As a result, it is well known that the patient may suffer with induction of hypomagnesemia, and frequency of tetany, arrhythmia, and convulsions may increase. On the contrary, infusion of the present preparation can maintain the magnesium concentration during the operation without additional magnesium administration, and decrease the occurrence of QT prolongation and arrhythmia. In a patient of intensive care unit (ICU) after surgical operation, tachyarrhythmia, particularly, atrial fibrillation and auricular flutter, frequently occur, and serum electrolyte imbalance is suggested. Then the intravenous injection of magnesium prevent of atrial fibrillation and auricular flutter, safely and effectively. That is, it is clinically important to maintain magnesium concentration in extracellular fluid, and therefore, the infusion of the preparation containing bicarbonate, having the maintenance and correction effect of magnesium concentration in extracellular fluid, prevents the hypomagnesemia which causes various kinds of diseases.

The BE indicated in the example of this specification means the index of the metabolic factors in acid-base equilibrium, and decreasing of BE means progress of metabolic acidosis. The progress of metabolic acidosis induces decrease of cardiac contractility and peripheral vasodilatation, and consequently, causes congestive heart failure, arterial hypotension and decreasing of blood flow volume. Further, the progress of metabolic acidosis causes the depression of threshold level of atrial fibrillation, which leads to fatal arrhythmia. Therefore, it is clinically important to improve the way to correct the procession of metabolic acidosis.

In the experiment test using dog hemorrhagic shock model in the Example 2, it was cleared that the preparation solution of the present invention rapidly increases bicarbonate level and BE, and the acidosis correction effect is superior to the reference solutions, such as Ringer's solution, lactated Ringer's solution, and acetated Ringer's solution. Additionally, bicarbonate level and BE were rapidly decreased after infusion of the present preparation as opposed to the bicarbonate level and BE of the reference solutions which were maintained or increased after administration.

In the experimental test using rabbit partial hepatectomy model, bicarbonate level and BE were decreased during the infusion of the reference solutions, such as Ringer's solution, lactated Ringer's solution, and acetated Ringer's solution. On the contrary, bicarbonate level and BE were maintained during the infusion of the preparation of the present invention, and decreased after the infusion. Therefore, the acidosis correction effect of the preparation of the present invention is superior to that of the referenced Ringer's solution, and the progress of acidosis caused by the partial hepatectomy can be delayed by administration of the preparation of the present invention.

The preparation containing bicarbonate of the present invention contains sodium bicarbonate as alkalizing agent in amount of 20 to 35 mEq/L, preferably 22 to 30 mEq/L. The solution also preferably contains 130 to 145 mEq/L of sodium ion, 2 to 5 mEq/L of potassium ion, 90 to 130 mEq/L of chloride ion, 2 to 5 mEq/L of calcium ion, 0.5 to 2.5 mEq/L of magnesium ion and 1 to 7 mEq/L of citrate ion as electrolytes, and 0 to 5 g/L of glucose. These electrolytes, for example, sodium chloride, sodium citrate, sodium acetate, sodium lactate, sodium dihydrogen phosphate, disodium hydrogen phosphate, sodium gluconate, sodium glycerophosphate, sodium malate, potassium chloride, dipotassium phosphate, potassium acetate, potassium citrate, potassium lactate, potassium glycerophosphate, potassium malate, calcium chloride, calcium lactate, calcium gluconate, calcium glycerophosphate, calcium hydrogen phosphate, calcium malate, magnesium chloride, magnesium gluconate, calcium glycerophosphate, and the like, are used without limitation where necessary.

The preferred components of the preparation containing bicarbonate are sodium chloride, potassium chloride, calcium chloride, magnesium chloride, sodium bicarbonate, sodium citrate and glucose.

Sodium bicarbonate, which is the resource of bicarbonate ion and plays a role of acid-base equilibrium for extracellular fluid, reacts with calcium ion or magnesium ion to form insoluble calcium carbonate or magnesium carbonate. Further, because the sodium bicarbonate solution releases carbon dioxide gas under heating or leaving to stand, and the pH of the solution increases, and it has been difficult to obtain the preparation solution stably containing bicarbonate ion.

Therefore, the preparation solution containing bicarbonate of the present invention may be preparation in situ, constituted by two-pack type solutions separately housed of sodium bicarbonate solution and electrolytes solution; however, one-pack type solution containing sodium bicarbonate and electrolytes is preferred for convenience in use.

### EXAMPLES

The present invention will be explained in detail by way of the following examples.

### Example 1

Using rabbit hemorrhagic shock model and partially hepatectomy model, the preparation solution containing bicarbonate with 20.0, 22.5, 25.0, 27.5 and 30.0 mEq/L in concentration were examined by comparison to acetated Ringer's solution.

The preparation of the present invention were prepared according to the formulations listed in Table 1.

Each component were dissolve in water to obtain 10 L of solution (pH: 8.0), and the resultant solution was bubbled with carbon dioxide gas to adjust pH to 6.7, and then filtrated. The obtained solution was filled into 500 mL glass vial and sterilized by high-pressure steam at 115° C. for 15 minutes. Thus, 5 preparation solutions containing bicarbonate with 20.0, 22.5, 25.0, 27.5 and 30.0 mEq/L in concentration (referred to as Test-22, Test-22.5, Test-25.0, Test-27.5 and Test-30.0, respectively) were obtained.

For the reference solution as acetated Ringer's solution (referred to as AR), Veen-F (Trade Mark) injection (Nikken Kagaku Co., Ltd.) was used.

TABLE 1

| | Conc. of HCO₃⁻ (mEq/L) | | | | |
|---|---|---|---|---|---|
| Components (g) | 20.0 | 22.5 | 25.0 | 27.5 | 30.0 |
| sodium chloride | 64.3 | 62.8 | 61.4 | 59.9 | 58.4 |
| potassium chloride | 2.98 | 2.98 | 2.98 | 2.98 | 2.98 |
| calcium chloride dihydrate | 2.21 | 2.21 | 2.21 | 2.21 | 2.21 |
| magnesium chloride hexahydrate | 1.02 | 1.02 | 1.02 | 1.02 | 1.02 |
| sodium bicarbonate | 16.8 | 18.9 | 21.0 | 23.1 | 25.2 |
| trisodium citrate dihydrate | 4.90 | 4.90 | 4.90 | 4.90 | 4.90 |

The pH value, in soluble foreign matters, in soluble particle number, contents of each component, and carbon dioxide gas concentration of space of these preparation solutions were measured at the start of examination and after storage for 3 months under room temperature.

The results are shown in Table 2.

TABLE 2

| | | | Insoluble | | | | | | | | | |
|---|---|---|---|---|---|---|---|---|---|---|---|---|
| | | Insoluble | particles | | | | | | | | | |
| conc. of | | foreign | (pieces/mL) | | Content (w/v %) | | | | | | | Space |
| HCO₃⁻ | pH | matter | 10 μm≧ | 25 μm≧ | Na | K | Ca | Mg | Cl | HCO₃ | citrate | (CO₂ %) |
| 20.0 mEq/L | | | | | | | | | | | | |
| Before | 7.2 | None | 0.0 | 0.0 | 0.302 | 0.015 | 0.00582 | 0.00114 | 0.4168 | 0.119 | 0.0309 | 7.16 |
| starting | | | | | | | | | | | | |
| 3 months | 7.1 | None | 0.5 | 0.0 | 0.302 | 0.015 | 0.00584 | 0.00115 | 0.4144 | 0.116 | 0.0318 | 9.85 |
| after | | | | | | | | | | | | |
| 22.5 mEq/L | | | | | | | | | | | | |
| Before | 7.1 | None | 0.1 | 0.0 | 0.304 | 0.015 | 0.00582 | 0.00116 | 0.4081 | 0.134 | 0.0310 | 5.40 |
| starting | | | | | | | | | | | | |
| 3 months | 7.2 | None | 0.9 | 0.0 | 0.303 | 0.015 | 0.00586 | 0.00111 | 0.4065 | 0.132 | 0.0319 | 9.53 |
| after | | | | | | | | | | | | |
| 25.0 mEq/L | | | | | | | | | | | | |
| Before | 7.1 | None | 0.0 | 0.0 | 0.303 | 0.015 | 0.00583 | 0.00116 | 0.4001 | 0.149 | 0.0309 | 6.10 |
| starting | | | | | | | | | | | | |
| 3 months | 7.1 | None | 0.4 | 0.0 | 0.303 | 0.015 | 0.00587 | 0.00112 | 0.3974 | 0.149 | 0.0320 | 10.24 |
| after | | | | | | | | | | | | |
| 27.5 mEq/L | | | | | | | | | | | | |
| Before | 7.1 | None | 0.0 | 0.0 | 0.302 | 0.015 | 0.00586 | 0.00116 | 0.3932 | 0.163 | 0.0310 | 8.02 |
| starting | | | | | | | | | | | | |
| 3 months | 7.2 | None | 0.2 | 0.0 | 0.305 | 0.015 | 0.00589 | 0.00113 | 0.3984 | 0.165 | 0.0322 | 10.42 |
| after | | | | | | | | | | | | |
| 30.0 mEq/L | | | | | | | | | | | | |
| Before | 7.1 | None | 0.0 | 0.0 | 0.304 | 0.015 | 0.00594 | 0.00115 | 0.3865 | 0.183 | 0.0312 | 10.14 |
| starting | | | | | | | | | | | | |
| 3 months | 7.2 | none | 0.1 | 0.0 | 0.306 | 0.015 | 0.00592 | 0.00114 | 0.3830 | 0.178 | 0.0322 | 11.90 |
| after | | | | | | | | | | | | |

As clearly shown by the results in the table, the preparation solutions of the present invention were stable and no degradation products and precipitations were observed after stored for 3 months.

a) Experiment Using Rabbit Hemorrhagic Shock Model

The rabbit hemorrhagic shock model was prepared by the following manner. The white male Japanese rabbits were anaesthetized with the combination of α-chloralose and urethane, and a cannula for blood discharging and blood sampling was placed in right femoral artery. After blood pressure and heart rate of rabbits were stabilized, the blood was discharged in order to obtain the blood pressure at about 40 mmHg. Then, the each preparation solution and reference solution were administered continuously at a rate of 60 mL/kg/hour for 90 minutes.

The blood pH, the plasma bicarbonate concentration and BE, which is the index of the variation of the metabolic factor in acid-base equilibrium, were measured before discharging of blood, and at 0 (just after start of the infusion), 15, 30, 60 and 90 (just after the infusion) minutes.

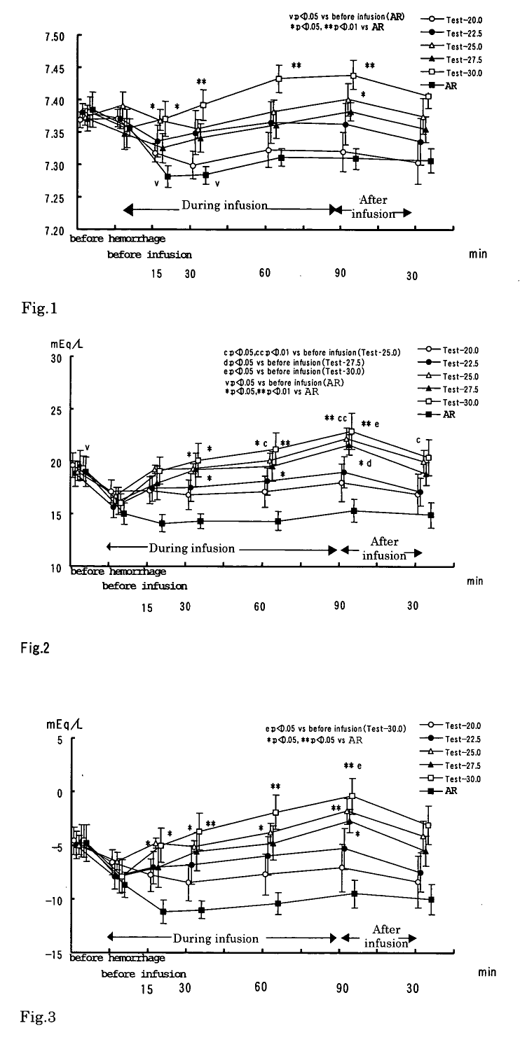

These results were shown in FIGS. 1 to 3.

The decreasing of pH value between 15 to 30 minutes after the start of the infusion was observed in the all the groups except Test-30.0, and subsequently, in the groups of Test-20.0 and the reference solution, the pH values stayed at a constant level. In the groups of Test-22.5, Test-25.0 and Test-27.5 showed the upward tendency of the pH values and the pH values were recovered to the level before the blood discharge. In the group of Test-30.0, the pH value increased over the level before the blood discharge.

No change of the plasma bicarbonate concentration was observed in the groups of Test-20.0 and the reference solution.

In the groups of the Test-22.5, Test-25.0, Test-27.5 and Test-30.0, the plasma bicarbonate concentration increased with the bicarbonate concentration of the preparation.

The change of BE level was observed in the similar manner as the change of the plasma bicarbonate concentration.

From these results, the acidosis correction effect of the preparation containing bicarbonate with concentration of 22.5, 25.0, 27.5 and 30.0 mEq/L was confirmed in rabbit hemorrhagic shock model.

b) Experiment Using Rabbit Partial Hepatectomy Model

Then, same experiments were conducted using rabbit partial hepatectomy model.

The rabbit partial hepatectomy model was prepared by the following manner. The white male Japanese rabbits were anaesthetized with pentobarbital, and a cannula was placed in trachea and rabbits were treated under anesthesia with isoflurane and respiratory management.

After blood pressure and heart rate of rabbits were stabilized, the each preparation solution and acetated Ringer' solution as the reference solution were continuously administered at a rate of 40 mL/kg/hour for 90 minutes. Just after the start of the infusion, abdominal operation was started and after 25 minutes, partial hepatectomy (75%) was conducted.

The blood pH, the plasma bicarbonate concentration and BE were measured at 0 (just after the start of the infusion), 15, 30, 60 and 90 (just after the completion of the infusion) minutes after starting of the infusion, and further 15 and 30 minutes after the completion of the infusion.

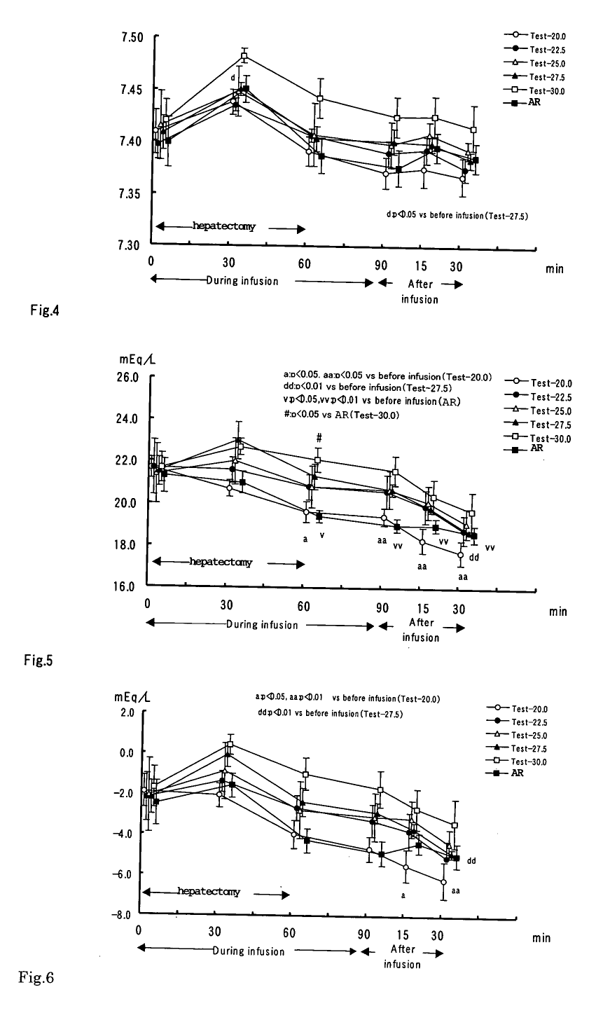

These results were shown in FIGS. 4 to 6.

In the group of Test-30.0, the pH value over the value before the infusion was observed during the infusion. On the contrary, in the groups of Test-20.0 and the reference solution, the pH value decreased during 60 to 90 minutes after the start of the infusion compared to the value before the infusion. In the groups of Test-22.5, Test-25.0 and Test-27.5, the intermediate pH values were observed.

The changes of the plasma bicarbonate concentration were observed in all of the groups, in proportion to the bicarbonate concentration of these preparations excluding the group of Test-20.0 in which the plasma bicarbonate concentration decreased below to that of before the infusion, as in the group of the reference solution.

The moderate decrease of the plasma bicarbonate concentration was observed in the groups of Test-22.5, Test-25.0 and Test-27.5, but these bicarbonate levels were recovered to that of before the infusion.

In the group of Test-30.0, the high plasma bicarbonate concentration in comparison with that of before the infusion was observed.

The change of BE level was observed in the similar manner as the changes of the plasma bicarbonate concentration.

From these results, the acidosis correction effect of the preparation containing bicarbonate with the concentration of 22.5, 25.0, 27.5 and 30.0 mEq/L was confirmed in rabbit partial hepatectomy model.

### Example 2

Using dog hemorrhagic shock model and rabbit partially hepatectomy model, the preparation of the present invention was examined by comparison to acetated Ringer's solution, lactated Ringer's solution and Ringer's solution.

The preparation was prepared according to the following formulations. That is, 61.4 g of sodium chloride, 2.98 g of potassium chloride, 2.21 g of calcium chloride dehydrate, 1.02 g of magnesium chloride hexahydrate, 21.0 g of sodium bicarbonate, and 4.90 g of sodium citrate were dissolve in water to obtain 10 L of solution (pH: 8.0). The resultant solution was bubbled with carbon dioxide gas to adjust pH to 6.7, and then filtrated. The obtained solution was filled in 500 mL glass vial and sterilized by high-pressure steam at 115° C. for 15 minutes. Thus, the preparation of the present invention (referred to as Test solution: TS) was obtained.

For the reference solution, Veen-F (Trade Mark) injection (Nikken Kagaku Co., Ltd.) was used as acetated Ringer's solution (AR), Solita (Trade Mark) (Shimizu) injection (Shimizu Pharmaceutical Co., Ltd.) was used as lactated Ringer's solution (referred to as LR) and Ringer's solution (Japanese Pharmacopoeia: Ohtsuka Pharmaceutical Co., Ltd.) was used as Ringer's solution (referred to as RS).

The pH value, insoluble particle number, contents of each component, and carbon dioxide gas concentration of space of the preparation were observed at the start of examination and after stored for 3 months at 25° C. Cunder60% of relative humidity. And the appearance of the precipitate was conducted by visual observation.

The results are shown in Table 3.

TABLE 3

| | | |
|---|---|---|
| insoluble | insolubleParticles | |
| foreign | (pieces/mL) | space |

| | pH | matter | 10 μm≧ | 25 μm≧ | (CO2%) |
|---|---|---|---|---|---|
| before | 7.0 | none | 0.4 | 0.0 | 11.73 |
| starting | | | | | |
| 3 months | 7.1 | none | 0.9 | 0.0 | 10.37 |
| after | | | | | |

| | content (w/v %) | | | | | | |
|---|---|---|---|---|---|---|---|
| | Na | K | Ca | Mg | Cl | HCO₃ | Citrate |
| before | 0.312 | 0.016 | 0.00606 | 0.00122 | 0.4025 | 0.152 | 0.0314 |
| starting | | | | | | | |
| 3 months | 0.313 | 0.016 | 0.00631 | 0.00121 | 0.4016 | 0.151 | 0.0321 |
| after | | | | | | | |

As clearly shown by the results in the table, the preparation of the present invention (Test solution) was stable one and no degradation products and precipitations were observed before and after the storage for 3 months.

a) Experiment Using Dog Hemorrhagic Shock Model

The male beagles were anaesthetized with pentobarbital, and treated under respiratory management using mixed gas (70% nitrogen/30% oxygen). The blood was discharged in a rate of 1 mL/kg/minute in order to obtain the average blood pressure at about 40 mmHg, and the blood was further discharged to maintain the blood pressure at about 40 mmHg when necessary, to obtain dog hemorrhagic shock model.

When BE of arterial blood was observed at −13 mEq/L, the preparation of the present invention or the reference solution was administered to the dog at a rate of 60 mL/kg/hour for 90 minutes.

The plasma bicarbonate concentration and BE were observed before the blood discharged, just after the blood discharge, and at 0 (just after start of the infusion), 15, 30, 60 and 90 (just after the completion of the infusion) minutes.

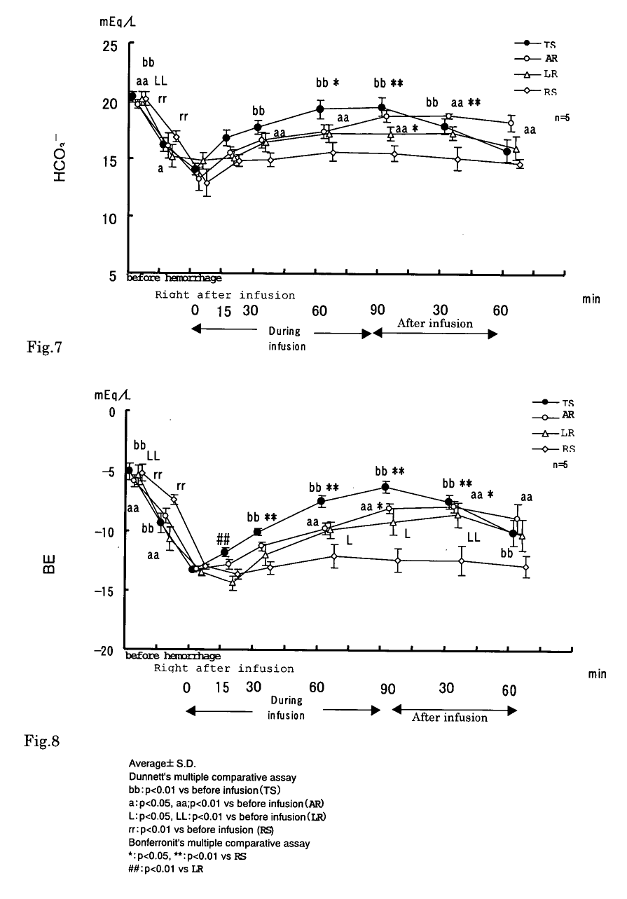

These results were shown in FIGS. 7 and 8.

The improvement effect for circular dynamic circular indicated by blood pressure and blood flow volume of the preparation of the present invention were same or superior in comparison with those of the reference solution. The acidosis correction effect of the present preparation was the best, and the preparation of the present invention corrected the acidosis by increasing the plasma bicarbonate concentration and BE rapidly.

Additionally, the acidosis correction effect of the preparation of the present invention disappeared promptly right after the infusion.

The degree of the serum magnesium concentration decrease was similar observed in the present preparation than the reference solutions.

b) Experiment Using Rabbit Partial Hepatectomy Model

The white male Japanese rabbits were anaesthetized with pentobarbital, and a cannula was placed in trachea and rabbits were treated under anesthesia with isoflurane and respiratory management.

After blood pressure and heart rate of rabbits were stabilized, the preparation solution of the present invention or the reference solution (acetated Ringer's solution, lactated Ringer's solution and Ringer's solution) was continuously administered at a rate of 40 mL/kg/hour for 90 minutes. Just after starting of the infusion, abdominal operation was started and after 25 minutes, partial hepatectomy (75%) was conducted.

The plasma bicarbonate concentration and BE were observed before the infusion, and at 30, 60 and 90 (just after the end of the infusion) minutes after start of the infusion, and further 15 and 30 minutes after the completion of the infusion.

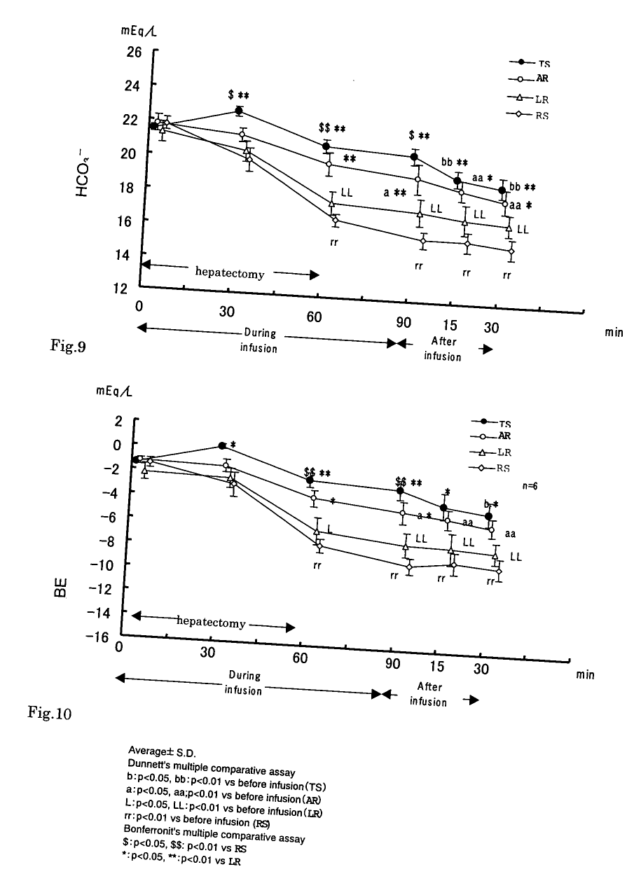

These results were shown in FIGS. 9 and 10.

The acidosis correction effect of the present preparation was the most excellent one and decrease the plasma bicarbonate concentration and BE were observed after infusion to delay the development of acidosis as opposed to the reference cases, where the decreasing of the plasma bicarbonate concentration and BE were observed during the infusion.

No decrease of the plasma magnesium concentration was observed in the case of the present preparation, as opposed to the case of the reference cases.

Industrial Applicability

As described above, the present invention provides the process for controlling water and electrolyte balance and acid-base equilibrium by means of administering of the preparation containing sodium bicarbonate as alkalizing agent, and particularly to process for controlling water and electrolyte balance and acid-base equilibrium supervening metabolic acidosis due to burn injury, hemorrhagic shock, multiple organ failure, systemic inflammatory response syndrome (SIRS), and so on. Namely, the preparation containing sodium bicarbonate can directly provide bicarbonate different from sodium lactate or sodium acetate which is metabolized to produce bicarbonate, and therefore this preparation is able to exhibit and disappear the acidosis correction effect quickly.

It is well known that the administration of organic acid salt, such as sodium lactate or sodium acetate and the like, may induce metabolic alkalosis because of bicarbonate production by metabolization after the completion of the administration thereof. Contrary, there is no anxiety about metabolic alkalosis by administration of the preparation containing sodium bicarbonate, because the acidosis correction effect disappears quickly. Further, the preparation containing bicarbonate, containing electrolytes in a balanced manner does not induce hypernatremia. Further, the preparation of the present invention possesses the maintenance and the correction effect of magnesium concentration in extracellular fluid.

1. A method for controlling water and electrolyte balance and acid-base equilibrium in a patient in need of such treatment, comprising administering continuously to the patient a preparation solution containing 130 to 145 mEq/L of sodium ion, 2 to 5 mEq/L of potassium ion, 20 to 35 mEq/L of bicarbonate ion, 90 to 130 mEq/L of chloride ion, 2 to 5 mEq/L of calcium ion, 0.5 to 2.5 mEq/L of magnesium ion, 1 to 7 mEq/L of citrate ion, and 0 to 5 g/L of glucose at a rate of 2 to 60 mL/kg/hour in an amount sufficient to control water and electrolyte balance and acid-base equilibrium in the patient.

2. A method for controlling water and electrolyte balance and acid-base equilibrium as claimed in claim 1, wherein data of blood gas analysis is observed as an index parameter of the water and electrolyte balance and acid-base equilibrium in the patient.

3. A method according to claim 15, wherein the infusion speed is adjusted in order to maintain a plasma bicarbonate concentration to be in a range of 22 to 26 mEq/L.

4. A method as claimed in claim 1, wherein said patient in need of such treatment suffers from metabolic acidosis.

5. A method as claimed in claim 1, wherein said patient in need of such treatment suffers from burn injury.

6. A method as claimed in claim 1, wherein said patient in need of such treatment suffers from hemorrhagic shock.

7. A method as claimed in claim 1, wherein said patient in need of such treatment suffers from multiple organ failure.

8. A method as claimed in claim 1, wherein said patient in need of such treatment suffers from systemic inflammatory reaction.

9. A method as claimed in claim 1, wherein said patient in need of such treatment is a patient undergoing an operation or is a post operative patient.

10. A method as claimed in claim 1, wherein said patient in need of such treatment suffers from hypohydremia.

11. A pharmaceutical composition for controlling water and electrolyte balance and acid-base equilibrium, comprising 130 to 145 mEq/L of sodium ion, 2 to 5 mEq/L of potassium ion, 20 to 35 mEq/L of bicarbonate ion, 90 to 130 mEq/L of chloride ion, 2 to 5 mEq/L of calcium ion, 0.5 to 2.5 mEq/L of magnesium ion, 1 to 7 mEq/L of citrate ion, and 0 to 5 g/L of glucose.

12. A pharmaceutical composition as claimed in claim 11, wherein said composition is in a form to be administered at a rate of 2 to 60 mL/kg/hour and maintain a plasma concentration of bicarbonate ion of 22 -26 mEq/L.

13. A pharmaceutical composition as claimed in claim 11, wherein a source of citrate ion is sodium citrate and pH of the agent is adjusted to 6.5 to 7.4 by carbon dioxide gas.

14. A pharmaceutical composition as claimed in claim 11, wherein said agent is filled in a carbon dioxide gas permeable plastic container sealed with gas un-permeable film, or in a gas un-permeable container.

15. A method as claimed in claim 2, wherein the infusion speed of administration or demedication of the preparation is adjusted based on the blood gas analysis.
